(19) United States
(12) Patent Application Publication
(10) Pub. No.: US 2006/0134193 A1
(43) Pub. Date: Jun. 22, 2006

(54) Method to improve pharmaceutical tablets having a matrix of cellulose ether

(76) Inventors: Pieter De Haan, Oss (NL); Cornelis Elisabeth Johannus Van Lare, Oss (NL)

Correspondence Address: F Aaro Dubberly;Akzo Nobel Inc, Intellectual Property Department, 7 Livingstone Avenue, Dobbs Ferry, NY, 10522-3408, US

(21) Appl. No.: 10/528,479

(22) PCT Filed: Sep. 17, 2003
(86) PCT No.: PCT/EP03/50627

§ 371 Date: Oct. 4, 2005

(30) Foreign Application Priority Data
Sep. 24, 2002 (EP) 02078968.1

(51) Int. Cl. A61K 9/20

(52) U.S. Cl. 424/464

(57) ABSTRACT

This invention provides for a packaged tablet, which tablet has a matrix consisting of at least 55% of a cellulose ether, whereby the tablet has a water activity of at most 0.6 and is packaged such as to delay moisture uptake by the tablet in order to decrease the vulnerability of the tablet to abrasion and attrition.

The invention relates to a packaged tablet, which tablet has a matrix consisting of at least 55% of a cellulose ether.

Such packaged tablets are generally known and usually the package is intended to protect the tablets from being polluted. Other characteristics of packaging can be intended to conveniently remove individual tablets from the package by the end users of the tablets.

Tablets having a matrix consisting of at least 55% of a cellulose ether turn out to be vulnerable to damage of the surface due to mechanical wear and tear. The problem is present from the moment in the production process that the tablets are leaving the tablet compression machine up to the moment at which such tablets are removed from the package and handheld by the person in need of using the medicine in the tablet. The problem is visible on conventionally packed tablets from powder in the bulk package of tablets, powder in bottles of tablets and powder in the single tablet pockets of a patient pack. The powder consists of loosened tablet material due to abrasion, which is wear of the tablets due to contact among the tablets or due to attrition, which is wear of the tablets due to contact against other material. Also, an unacceptable amount of dust forms during processing the tablets, for example, during manual or automatic blistering.

Tablets of the defined characteristics can be prepared by dry-mixing processes whereby the tablets are manufactured by direct compression of a powder mixture or by compressing a preformed granulate into a tablet without the use of granulation liquids.

A known method to solve a problem of dust formation is treating the tablet surface with coating materials and thereby adding a layer of another composition to the tablets. The purpose of this invention is to reduce the dust formation during handling the tablets without using any additional coating materials.

Testing of tablets conditioned to different humidity conditions showed a reduction in dust formation with decreasing relative humidity storage condition (thus decreasing moisture content).

It has now been found that there is reduced dust formation with packaged tablets which have a water activity of at most 0.6 and the tablets are packaged such as to delay moisture uptake by the tablets. Preferred are packaged tablets which have a water activity of at most 0.55, but lower water activity, such as 0.50 or even less than 0.45, is preferred. Such packaged tablets have decreased vulnerability to abrasion and attrition.

The term water activity is used as is known from the thermodynamic concept of activity of chemical compounds. The activity is defined here to be measured at 25° C. and 1 Atmosphere. The term is a quantitative term describing the availability of water for any chemical interaction. In pharmaceutics it is commonly used in sorption isotherms which describe the relation between water content of a product and the corresponding relative humidity (RH) of the air in equilibrium with the product at that water content. The equilibrium RH is directly correlated to the water activity, that is: Water Activity=RH/100. In tablets having about 80% hydroxypropylmethylcellulose as matrix substance, a water activity of 0.6 in the tablets corresponds approximately to 9.0% w/w water content. The latter water content is defined as the content of water determined by the Karl-Fischer method, implying that this water content includes, for example, the amount of crystal water of the ingredients of the tablet. Thus the invention provides for packaged tablets having less than 9 w/w % water content having reduced dust formation.

Tablets having a matrix consisting of at least 55% of a cellulose ether are the kind of tablets with the cumbersome dust forming surface if not treated with the method according to the invention. It is the high content of the cellulose ether and the properties of the matrix which cause the tablets having such a vulnerable surface. Since the problem of dust formation with tablets having a matrix consisting of a cellulose ether can also be influenced by the nature and amounts of other constituents, such as the filler or binder and active ingredient, the method according to the present invention is preferrably to be applied to tablets having a matrix which consists of at least 65% of the cellulose ether and more preferrably of from 70 to 85 wt % of the cellulose ether. Also, a reduced amount of carbohydrate binder, such as Avicel, which is microcrystalline cellulose, can be problematic for untreated tablets. More specifically, for example less than 15% and in particular less than 10 wt % is problematic for untreated tablets. Tablets with carbohydrate binders are for example cellulose (7 to 10 wt % microcrystalline cellulose, such as Avicel pH 101), sugars, starches, amylopectin, dextrin, maltodextrin, gums and alginates.

After preparing the tablets under the suitable low water activity condition the water content should be maintained at reduced level by protecting the tablets from environmental moisture, hence the recommended packaging having the property to delay moisture uptake by the tablets. Such packaging is well known in the art of pharmaceutical packaging. The Remington; The Science and Practice of Pharmacy; 20th ed., Publisher: Lippincott Williams & Wilkins; Baltimore; USA in Chapter 54: Plastic packaging materials, under the heading of mass transfer on page 1006. Packaging materials are available which protect tablets adequately from water vapor, providing a barrier to moisture, thereby hampering transfer of water towards the tablets in the package. Examples of packages having the property to delay moisture uptake by the tablets are containers that are closed by sealing, or blistered tablets in an aluminium sachet. It is therefore another aspect of this invention to provide a patient pack comprising one or more tablets having a matrix consisting of at least 55% of a cellulose ether whereby the tablet has a water activity of at most 0.6 and the package is such as to delay moisture uptake by the tablets. Other specific examples of packaged tablets according to the invention are bottles of 75 cc made of amber glass with 38 mm closure and comprising a canister or sachet with a desiccant and containing 30-50 tablets per bottle. Another embodiment is a capped and sealed high density polyethylene (HDPE) bottle with desiccant canister. The closures can be lined with an inner seal consisting of pulpboard/wax/foil laminate which is affixed to the inner cap. Packaged tablets according to the invention can also be in a can or in aluminium-aluminium blister package or in a normal blister package which is further provided with an Aclar® film. Aclar is a flexible material made from fluorinated chlorinated resins, such as, e.g., Aclar22®, which is a polymer from chlorotrifluoroethene and 1,1-difluoroethene monomers. The use of polyvinyldichloride (PVDC) or poly-ethyleneterephtalate (PET) are useful packaging materials to delay moisture uptake by the tablets.

Cellulose ethers are used as carrier in dry-mix tablets, as binder in wet-granulation and can be used in coating techniques as film-forming polymers. Such carriers tend to retain in aquous environment other ingredients for a longer time upon absorption of water in the outer layer and consequently are suitable for extended release formulation. Examples of such carriers can be found in the group of hydroxy-(1C-3C)alkyl(1C-3C)alkylcelluloses, such as hydroxymethylcellulose, hydroxyethylcellulose and the preferred hydroxypropylmethylcellulose (HPMC). Other gel-forming carriers can be found in the standard compilation of pharmaceutically acceptable carriers and excipients, the Handbook of Pharmaceutical Excipients (3nd edition edited by Arthur H. Kibbe; Published by the American Pharmaceutical Association, Washington D.C. and The Pharmaceutical Press, London in 2000).

The cellulose ethers and the relevant theoretical views on their properties are discussed in Alderman, A., A review of cellulose ethers in hydrophilic matrices for oral controlled-release dosage forms, Int. J. Pharm. Tech. & Prod. Mfr., Vol 5, pages 1-9, 1984.

The tablets for which the present invention can be used can have a total weight of at most 450 mg and may have a high relative amount of the active ingredient gepirone HCl, e.g. 60, 80, or up to 85 mg gepirone HCl, over the cellulosic polymer matrix material and also over the carbohydrate binder. With the present invention such tablets could still be used without unacceptable dust formation during handling.

Tablets having the composition as shown in Table 1 were prepared.

TABLE 1
Composition of example tablets; quantities are in mg

| | 20 | 40 | 60 | 80 | |
|---|---|---|---|---|---|
| Ingredients | mg | mg | mg | mg | Function |
| Gepirone HCl | 20.0 | 40.0 | 60.0 | 80.0 | Active principle |
| Hydroxypropyl | 290.0 | 290.0 | 290.0 | 290.0 | Drug release |
| Methylcellulose | | | | | controlling polymer |
| (Methocel K100M, | | | | | |
| Premium | | | | | |
| Microcrystalline | 61.8 | 52.12 | 31.0 | 33.7 | Diluent |
| Cellulose (Avicel | | | | | |
| pH 101) | | | | | |
| Euroxide, yellow ferric | 0.40 | 0.08 | 1.2 | 3.5 | Colorant |
| oxide and/or red ferric | | | | | |
| oxide | | | | | |
| (E7055, E7056 or | | | | | |
| E7016) | | | | | |
| Colloidal anhydrous | 1.60 | 1.60 | 1.60 | 1.60 | Glidant |
| silicon dioxide | | | | | |
| (cab-o-sil M5) | | | | | |
| Magnesium stearate | 1.20 | 1.20 | 1.20 | 1.20 | Lubricant |
| NF | | | | | |

Active Pre-Mixture:

Transfer the colloidal silicon dioxide, NF, colorant (40 mg: Euroxide Yellow E 7056; 60 mg: Euroxide Yellow E 7055; 80 mg: Euroxide yellow E 7055 and Euroxide Red E 7016), gepirone HCl powder and 20% of hydroxypropyl methylcellulose USP in 2 cu. Ft. planetary mixer (Hobart mixer). Mix ingredients for 15 minutes in a planetary mixer (Hobart Mixer). Label as ‘Active Pre-Mix’.

Blend for Slugging

Mill the Active Pre-Mix in a Fitzmill using a perforated plate No. 0020 at high speed, impact forward to deagglomerate lumps, if any.

Transfer the Active Pre-Mix in a 10 cu. Ft. “V”-blender without an I-bar, while passing through #12 mesh screen and transfer the balance of 80% HPMC, microcrystalline cellulose, NF and 50% of magnesium stearate, NF in the V-blender without an I-bar. Blend ingredients in the V-blender without an I bar for 24 minutes and label as “Blend for Slugging”.

Slugging

Compress the blend into slugs using ⅞″ round flat face tooling using a rotary Kikusui-Hercules compression machine.

In Process Controls:

| Weight: | 2250 mg |
|---|---|
| Hardness: | 7 kp |
| Targeted thickness: | 0.255″ |

Final Blend

Mill the slugs in an S.S. Fitzmill with screw feeder using a perforated plate No. 0093 at medium speed, knives forward and screw feeder setting of 3.5 ±0.5. Transfer the milled mass into a 10 cu. Ft. S.S. V-blender without I-bar. Screen the balance of 50% magnesium stearate, NF through # 18 mesh and transfer also into the V-blender. Blend for 6 minutes.

Compress tablets with a rotary Kikusui-Libra compression machine using 0.338″ X 0.405″ Ovoid rectangular dies.

In Process Controls:

| | | |
|---|---|---|
| Strength, mg | 40 & 60 | 80 |
| Run Weight, mg | 385 ± 27 | 410 ± 29 |
| Hardness, kp | 18 ± 4 | 20 ± 8 |
| Thickness, inches | 0.230 to 0.260 | 0.235 to 0.265 |

Testing for vulnerability of the tablets to abrasion and attrition.

Tablets of two different strengths of gepirone HCL with compositions according to Table 1 were conditoned for 1 week to defined relative humidities in order to equilibrate the tablet to acquire different water activities. After this the tablets were tested for dust determination with the following method: 10 tablets are placed for 45 minutes in a Securitainer of Ø49 x h 58 mm and shaken using a vibrating table at 200 rpm with a horizontal amplitude of 45 mm. The mass loss of the tablets is determined by weighing. Before weighing the tablets are cleaned by vacuum air. The water content of tablets conditioned to the various water activities was determined by adding the water content before conditioning, as determined by the method according to Karl-Fischer, to the weight increase due to water uptake after conditioning of the tablets in the various relative humidities at temperature of 25° C. and 1 atmosphere pressure.

TABLE 2
Average percentages dust formed using shaking table method.

| | | | Water content |
|---|---|---|---|
| Gepirone content | Water | Dust formed in % | in % w/w of |
| of tablet | activity | w/w of tablet weight | tablet weight |
| 20 mg | 0.43 | 1.65 | 6.4 |
| 20 mg | 0.60 | 2.01 | 9.0 |
| 20 mg | 0.75 | 3.42 | 11.7 |
| 80 mg | 0.43 | 2.11 | 4.7 |
| 80 mg | 0.60 | 2.93 | 7.2 |
| 80 mg | 0.75 | 4.33 | 9.7 |

The results demonstrate that the vulnerability for dust formation increases with increasing water activity in the tablets. A significant increase in dust formation occurs at water activities higher than 0.6.

1. A packaged tablet, which tablet has a matrix consisting of at least 55% of a cellulose ether, wherein the tablet has a water activity of at most 0.6 and is packaged such as to delay moisture uptake by the tablet.

2. The packaged tablet according to claim 1, wherein the tablet has a water activity of less than 0.55.

3. A packaged tablet, which tablet has a matrix consisting of at least 55% of a cellulose ether, wherein the tablet has a water content of less than 9% w/w and is packaged such as to delay moisture uptake by the tablet.

4. The packaged tablet according to claim 1, wherein the matrix consists of more than 65% of a cellulose ether.

5. The packaged tablet according to claim 4, wherein the cellulose ether is hydroxypropyl methylcellulose.

6. The packaged tablet according to claim 1, wherein the tablet comprises gepirone HCl in an amount in the range of from 20-85 ma.

7. The packaged tablet according to claim 3, wherein the matrix consists of more than 65% of a cellulose ether.

8. The packaged tablet according to claim 3, wherein the tablet comprises gepirone HCl in an amount in the range of from 20-85 ma.

9. The packaged tablet according to claim 1, wherein the tablet comprises gepirone HCl in an amount in the range of from 20-85 ma and the matrix consists of more than 65% of a cellulose ether.

10. The packaged tablet according to claim 9, wherein the cellulose ether is hydroxypropyl methylcellulose.
